(19) United States
(12) Patent Application Publication
(10) Pub. No.: US 2024/0197665 A1
(43) Pub. Date: Jun. 20, 2024

(54) Pharmaceutical Composition For Treating Hyperlipidemia And Preparation Method Therefor

(71) Applicant: SICHUAN JIUZHANG BIOLOGICAL SCIENCE AND TECHNOLOGY CO., LTD., Chengdu, Sichuan (CN)

(72) Inventors: Jie ZHANG, Chengdu, Sichuan (CN); Wenbin LI, Chengdu, Sichuan (CN); Wang HUANG, Chengdu, Sichuan (CN); Fei ZHANG, Chengdu, Sichuan (CN); Ya ZHANG, Chengdu, Sichuan (CN); Min XU, Chengdu, Sichuan (CN)

(21) Appl. No.: 18/556,818

(22) PCT Filed: May 9, 2022
(86) PCT No.: PCT/CN2022/091766

§ 371 Date: Oct. 23, 2023

(30) Foreign Application Priority Data
May 11, 2021 (CN) 202110509462.6

(51) Int. Cl. A61K 31/216; A61K 31/355; A61K 31/375; A61K 33/04; A61K 33/24; A61K 33/30; A61K 33/32; A61P 3/06

(52) U.S. Cl. CPC A61K 31/216; A61K 31/355; A61K 31/375; A61K 33/04; A61K 33/24; A61K 33/30; A61K 33/32; A61P 3/06

(57) ABSTRACT

A pharmaceutical composition for treating hyperlipidemia and a preparation method therefor are provided. The pharmaceutical composition contains chlorogenic acid, vitamins, pharmaceutically acceptable excipients and optional microelements. The pharmaceutical composition for treating hyperlipidemia reduce can serum total cholesterol and serum triglyceride, and increase the content of high-density lipoproteins.

### TECHNICAL FIELD

The present invention belongs to the medicinal field, and specifically relates to a pharmaceutical composition for treating hyperlipidemia and a preparation method therefor.

### BACKGROUND TECHNOLOGY

Hyperliproteinemia refers to the increase of cholesterol and triglyceride levels in plasma as well as the decrease of high-density lipoprotein (HDL) levels. It is one of the important factors that cause atherosclerosis, coronary heart disease, hypertension, diabetes, stroke, and the same. At present, there is no unified diagnostic standard for hyperlipidemia both domestically and internationally. Generally, when the plasma total cholesterol concentration is >5.17 mmol/L (200 mg/dl), the patient can be diagnosed as having hypercholesterolemia, and when the plasma triglyceride concentration is >2.3 mmol/L (200 mg/dl), the patient can be diagnosed as having hypertriglyceridemia.

Hyperlipidemia can be divided into two types: primary hyperlipidemia and secondary hyperlipidemia. Primary hyperlipidemia is caused by congenital defects in lipid and lipoprotein metabolism, while secondary hyperlipidemia is mostly secondary to metabolic disorders such as diabetes, hypertension, liver disease, kidney disease, thyroid disease, drinking, smoking, high-fat diet, etc.

At present, the treatments of hyperlipidemia include non-drug therapy and drug therapy, and for the non-drug therapy, the methods such as weight control, exercise, smoking cessation, and dietary therapy are generally used to treat hyperlipidemia, while for drug therapy, statins that can reduce the levels of serum total cholesterols and low-density lipoproteins, or other medicaments such as fibrates and niacins that can reduce serum triglyceride levels are used.

Chlorogenic acid is a phenylpropanoid compound synthesized from the intermediate product of the pentose phosphate pathway during aerobic respiration in plants. Its extraction technology is mature and reliable, and it can also be synthesized in small amounts. Chlorogenic acid has been developed and applied in various fields such as food, health products, cosmetics, and pharmaceuticals. The current research results indicate that chlorogenic acid has many pharmacological effects such as cardiovascular protection, antioxidant, anti-UV, anti-radiation, anticancer, antibacterial, antiviral, immune regulation, and treating metabolic disorders.

It has been reported that vitamins such as vitamin E and vitamin C, as well as trace elements such as zinc, copper, manganese, iron, chromium, selenium, calcium, magnesium, etc., are all related to lipid metabolism. Based on this, some scholars have proposed to regulate lipid metabolism levels by supplementing trace elements. However, the effects of vitamins and trace elements on regulation of blood lipids are not soundly clear, and excessive supplementation of vitamins and trace elements may also lead to various health problems. Therefore, there have been no clinical reports on the treatment of hyperlipidemia by supplementing vitamins and trace elements.

The present invention is intended to providing a pharmaceutical composition with excellent therapeutic effects on hyperlipidemia.

### SUMMARY OF THE INVENTION

The present invention aims to provide a pharmaceutical composition for treating hyperlipidemia, as well as a preparation method therefor, and uses thereof.

In one aspect, the present invention provides a pharmaceutical composition for treating hyperlipidemia, which comprises chlorogenic acid, vitamins, pharmaceutically acceptable excipients, and optional trace elements.

Preferably, the vitamin is selected from: vitamin C, vitamin E, vitamin B6, vitamin B12 or a combination thereof; more preferably, the vitamin is selected from vitamin C;
preferably, the trace element is selected from: zinc, manganese, selenium, chromium, copper or a combination thereof; more preferably, the trace element is selected from zinc gluconate, manganese gluconate, chromium chloride, copper gluconate or a combination thereof; most preferably, the trace element is selected from manganese gluconate;
preferably, the pharmaceutically acceptable excipients are selected from: bulking agents, adhesives, disintegrating agents, lubricants, solvents, antioxidants, stent agents, etc; more preferably, the antioxidant is selected from sodium bisulfite, sodium pyrosulfite, L-cysteine hydrochloride, and a combination thereof, as well as the stent agent is selected from sucrose, mannitol, glucose, lactose, trehalose, hydroxyethyl starch, dextran 20, sorbitol, PEG1000, glycerol, glycine, 1,2-propanediol, and a combination thereof;
preferably, the pharmaceutical composition for treating hyperlipidemia comprises chlorogenic acid, vitamins, and optional trace elements as the only active ingredients;
preferably, the weight ratio of chlorogenic acid, vitamins, and optional trace elements is 100:(1-5):(0.01-0.05); preferably, the weight ratio of chlorogenic acid, vitamins, and optional trace elements is 100:(2-4):(0.02-0.04); more preferably, the weight ratio of chlorogenic acid, vitamins, and optional trace elements is 100:3:0.03;

The pharmaceutical composition for treating hyperlipidemia according to the present invention is administered by a gastroenteric or parenteral route, and more preferably, the pharmaceutical composition for treating hyperlipidemia according to the present invention is administered by a parenteral route.

Preferably, the pharmaceutical formulation for gastrointestinal administration of the pharmaceutical composition for treating hyperlipidemia according to the present invention includes: tablets, capsules, granules, and oral liquids, while the pharmaceutical formulation for parenteral administration includes: injection, infusion solution, and freeze-dried powder injections;

In another aspect, the present invention provides a preparation method of the pharmaceutical composition for treating hyperlipidemia, which comprises the following steps:
(1) Each raw material is weighed according to the pre-determined amount;
(2) A pharmaceutical formulation for administration by the gastrointestinal or parenteral pathways is prepared according to conventional methods in this field;
specifically, the present invention provides a preparation method of an injection for treating hyperlipidemia, comprising the following steps:
(1) Each raw material is weighed according to the pre-determined amount;
(2) To water for injection, are successively added chlorogenic acid, antioxidants, vitamins, and optional trace elements, and then the mixture is stirred until all of the agents are fully dissolved, and then the resultant solution is diluted to the required volume. The pH is adjusted to be 2-5, and then activated carbon is added, followed by stirring for 20-40 min. The solution is filtered to remove the activated carbon, and then the solution is further filtered with a 0.22 μm hydrophilic microporous filter membrane until the filtrate is clear, which is aseptically filled into the suitable container;
The present invention provides a preparation method of a freeze-dried powder injection for treating hyperlipidemia, comprising the following steps:
(1) Each raw material is weighed according to the pre-determined amount;
(2) To water for injection, are successively added chlorogenic acid, antioxidants, stent agents, vitamins, and optional trace elements, and then the mixture is stirred until all of the agents are fully dissolved, and then the resultant solution is diluted to the required volume. The pH is adjusted to be 2-5, and then activated carbon is added, followed by stirring for 20-40 min. The solution is filtered to remove the activated carbon, and then the solution is further filtered through a 0.22 μm hydrophilic microporous filter membrane until the filtrate is clear, which is aseptically filled into a suitable container and freeze-dried;
The present invention provides a preparation method of a tablet for treating hyperlipidemia, comprising the following steps:
(1) Each raw material is weighed according to the pre-determined amount;
(2) Chlorogenic acid, vitamins, optional trace elements, bulking agents, and disintegrating agents are passed through a 60-100 mesh sieve and fully mixed. A suitable amount of adhesive is added to make soft materials, which is granulated by passing through a 14 mesh sieve, dried, and passed through a 12 mesh sieve for breaking. Lubricants are added and mixed evenly, followed by pressing to obtain tablets.

In another aspect, the present invention provides the combination of chlorogenic acid, vitamins, and optional trace elements for use in the manufacture of a pharmaceutical composition for treating hyperlipidemia;
preferably, the vitamin is selected from: vitamin C, vitamin E, vitamin B6, vitamin B12 or a combination thereof; more preferably, the vitamin is selected from vitamin C;
preferably, the trace element is selected from: zinc, manganese, selenium, chromium, copper, etc.; more preferably, the trace element is selected from zinc gluconate, manganese gluconate, chromium chloride, copper gluconate or a combination thereof; most preferably, the trace element is selected from manganese gluconate;
preferably, the pharmaceutically acceptable excipients are selected from: bulking agents, adhesives, disintegrating agents, lubricants, solvents, antioxidants, stent agents, etc; more preferably, the antioxidant is selected from sodium bisulfite, sodium pyrosulfite, L-cysteine hydrochloride, and a combination thereof, as well as the stent agent is selected from sucrose, mannitol, glucose, lactose, trehalose, hydroxyethyl starch, dextran 20, sorbitol, PEG1000, glycerol, glycine, 1,2-propanediol, and a combination thereof;
preferably, the pharmaceutical composition for treating hyperlipidemia comprises chlorogenic acid, vitamins, and optional trace elements as the only active ingredients;
preferably, the weight ratio of chlorogenic acid, vitamins, and optional trace elements is 100:(1-5):(0.01-0.05); more preferably, the weight ratio of chlorogenic acid, vitamins, and optional trace elements is 100:(2-4):(0.02-0.04); most preferably, the weight ratio of chlorogenic acid, vitamins, and optional trace elements is 100:3:0.03;

The pharmaceutical composition for treating hyperlipidemia according to the present invention is administered by a gastroenteric or parenteral route, and more preferably, the pharmaceutical composition for treating hyperlipidemia according to the present invention is administered by a parenteral route.

Preferably, the pharmaceutical formulation for gastrointestinal administration of the pharmaceutical composition for treating hyperlipidemia according to the present invention includes: tablets, capsules, granules, oral liquids, etc., while the pharmaceutical formulation for parenteral administration includes: injection, infusion solution, freeze-dried powder injections, etc.

The beneficial effects of the present invention:

In the present invention, a pharmaceutical composition with excellent therapeutic effects on hyperlipidemia is obtained by combining chlorogenic acid with vitamins and optional trace elements, and especially, the combination of chlorogenic acid, vitamin C, and manganese gluconate has the most excellent effect, which can significantly reduce the levels of serum total cholesterol and triglyceride, and increase the level of high-density lipoproteins. The pharmaceutical composition for treating hyperlipidemia according to the present invention has clear components, simple composition, and exact lipid-lowering effects, as well as is safe without side effects, and thus can be widely used in the clinical treatment of hyperlipidemia.

### EXAMPLES

The present invention had been illustrated in greater detail below, to aid in the understanding of the present invention.

### Example 1: An Injection for Treating Hyperlipidemia

100 g of chlorogenic acid, 3 g of vitamin C, 1 g of sodium bisulfite, 0.03 g of manganese gluconate, and a suitable amount of water for injection were processed as follows:
(1) Each raw material was weighed according to the pre-determined amount;
(2) To water for injection, were successively added chlorogenic acid, sodium bisulfite, vitamin C, and manganese gluconate, and then the mixture was stirred until all of the agents were fully dissolved. The pH of the resultant solution was adjusted to be 3.5, and then diluted to 2000 mL. Activated carbon was added, and then the solution was stirred for 20-40 min, followed by filtration to remove the activated carbon. The solution was further filtered through a 0.22 μm hydrophilic microporous filter membrane until the filtrate was clear, which was aseptically filled into a container.

### Example 2: A Freeze-Dried Powder Injection for Treating Hyperlipidemia

100 g of chlorogenic acid, 4 g of vitamin C, 2 g of sodium bisulfite, 0.05 g of manganese gluconate, 80 g of mannitol, and a suitable amount of water for injection were processed as follows:
(1) Each raw material was weighed according to the pre-determined amount;
(2) To water for injection, were successively added chlorogenic acid, sodium bisulfite, mannitol, vitamin C, and manganese gluconate, and then the mixture was stirred until all of the agents were fully dissolved. The pH of the resultant solution was adjusted to be 4, and then diluted to 2000 mL. Activated carbon was added, and then the solution was stirred for 20-40 min, followed by filtration to remove the activated carbon. The solution was further filtered through a 0.22 μm hydrophilic microporous filter membrane until the filtrate was clear, which was aseptically filled to obtain 1000 injections, and then freeze-dried, to provide freeze-dried powder injections.

### Example 3: Tablets for Treating Hyperlipidemia

100 g of chlorogenic acid, 3 g of vitamin C, 0.03 g of manganese gluconate, 364 g of microcrystalline cellulose, 30 g of cross-linked sodium carboxymethyl cellulose, 3 g of magnesium stearate, and a suitable amount of ethanol were processed as follows:
(1) Each raw material was weighed according to the pre-determined amount;
(2) Chlorogenic acid, manganese gluconate, vitamin C, microcrystalline cellulose, and cross-linked sodium carboxymethyl cellulose were passed through an 80 mesh sieve and fully mixed. A suitable amount of ethanol was added to make soft materials, which was granulated through a 14 mesh sieve, dried, and passed through a 12 mesh sieve for breaking. Magnesium stearate was added and mixed evenly, followed by pressing to provide 1000 tablets for treating hyperlipidemia.

### Experimental Example 1: The Effect of Reducing Blood Lipid Level for the Pharmaceutical Composition for Treating Hyperlipidemia According to the Present Invention

### 1.1 Experimental Drugs

| Groups | Experimental drugs |
|---|---|
| 1 | chlorogenic acid |
| 2 | chlorogenic acid:vitamin C = 20:1.2 |
| 3 | chlorogenic acid:vitamin E = 20:1.2 |
| 4 | chlorogenic acid:vitamin C:vitamin E = 20:0.6:0.6 |
| 5 | chlorogenic acid:zinc gluconate = 20:1.2 |
| 6 | chlorogenic acid:manganese gluconate = 20:1.2 |
| 7 | chlorogenic acid:chromium chloride = 20:1.2 |
| 8 | chlorogenic acid:zinc gluconate:manganese |
| | gluconate = 20:0.6:0.6 |
| 9 | chlorogenic acid:zinc gluconate:chromium chloride = 20:0.6:0.6 |
| 10 | chlorogenic acid:manganese gluconate:chromium |
| | chloride = 20:0.6:0.6 |
| 11 | chlorogenic acid:zinc gluconate:manganese gluconate:chromium |
| | chloride = 20:0.4:0.4::0.4 |
| 12 | chlorogenic acid:vitamin C:zinc gluconate = 20:0.6:0.6 |
| 13 | chlorogenic acid:vitamin C:manganese gluconate = 20:0.6:0.6 |
| 14 | chlorogenic acid:vitamin C:chromium chloride = 20:0.6:0.6 |
| 15 | chlorogenic acid:vitamin C:zinc gluconate:manganese |
| | gluconate = 20:0.4:0.4:0.4 |
| 16 | chlorogenic acid:vitamin C:zinc gluconate:chromium |
| | chloride = 20:0.4:0.4:0.4 |
| 17 | chlorogenic acid:vitamin C:manganese gluconate:chromium |
| | chloride = 20:0.4:0.4:0.4 |
| 18 | chlorogenic acid:vitamin C:zinc gluconate:manganese |
| | gluconate:chromium chloride = 20:0.3:0.3:0.3:0.3 |

The above drugs were formulated as injections or emulsions according to the method of Example 1, in which the total concentration of the active drugs was adjusted to 0.53%, and the emulsion was uniformly mixed prior to use.

### 1.2 Experimental Animals

After one day of adaptive feeding, 210 male Kunming mice weighing 18-20 g were randomly divided into 21 groups, with 10 mice in each group, including the normal group, the model group, the blank group, and drugs 1-18 groups, and the day was recorded as the first day of the experiment. The mice in the normal group were fed by standard diet, while the mice in the model group, the blank group, and drugs 1-18 groups were all fed by high-fat diet, in which the formula of the standard diet was: corn flour 60%, bran 35%, flour 2%, fish meal 1.5%, salt 1%, and fish liver oil 0.5%, and the formula of the high-fat diet was: cholesterol 2%, sodium cholate 0.5%, lard 7.5%, and standard diet 90%. All mice were allowed to drink freely. Each group of mice were injected with the corresponding experimental drug via the tail vein twice a day, 200 μL for each time. The normal group and the blank group were injected with the same amount of injection water via the tail vein. The mice were continuously fed for 4 weeks, in which the mice were weighed on the 6th, 13th, 20th, and 27th days after no drinking water for 8 hours. On the 28th day, 2 hours after the last administration of the drug, blood was collected from the orbit of the mice. The levels of the total cholesterol, triglycerides, and high-density lipoproteins in serum were measured using ELISA. After the mice were euthanized by cervical dislocation, the intact liver was retrieved and washed with 0-4° C. physiological saline, which was wiped dry with filter paper, and then the liver index was calculated. The specific experimental results are shown in Tables 1-2, wherein:

Liver index=liver wet weight/body weight*100.

### 1.3 Experimental Results

The multi-way ANOVA module of statistical software SPSS was used for data analysis, in which P<0.05 indicates the result is statistically different, while P<0.01 indicates the result is significantly different.

TABLE 1
The effect of the pharmaceutical composition of the present
invention on the body weight of mice having a high-fat diet.

| | Body weight (g) | | | | |
|---|---|---|---|---|---|
| | Initial body | | | | |
| Groups | weight | Day 6 | Day 13 | Day 20 | Day 27 |
| Blank | 19.53 ± 0.13 | 21.35 ± 0.22 | 23.37 ± 0.29 | 24.67 ± 0.18 | 27.16 ± 0.24 |
| group | | | | | |
| Model | 19.15 ± 0.14 | 22.62 ± 0.31 | 25.48 ± 0.33Δ | 27.05 ± 0.36Δ | 29.13 ± 0.31Δ |
| group | | | | | |
| 1 | 19.36 ± 0.16 | 22.51 ± 0.28 | 25.11 ± 0.26 | 26.76 ± 0.34 | 28.07 ± 0.21 |
| 2 | 19.72 ± 0.21 | 22.75 ± 0.32 | 24.97 ± 0.30 | 26.14 ± 0.25 | 27.86 ± 0.34 |
| 3 | 19.52 ± 0.16 | 23.32 ± 0.27 | 25.34 ± 0.34 | 26.85 ± 0.31 | 28.18 ± 0.41 |
| 4 | 19.13 ± 0.19 | 22.97 ± 0.24 | 25.15 ± 0.29 | 26.52 ± 0.35 | 27.67 ± 0.37 |
| 5 | 19.73 ± 0.22 | 23.34 ± 0.31 | 25.69 ± 0.33 | 27.28 ± 0.37 | 28.24 ± 0.41 |
| 6 | 19.64 ± 0.27 | 23.27 ± 0.26 | 25.37 ± 0.31 | 26.51 ± 0.29 | 27.66 ± 0.35 |
| 7 | 19.65 ± 0.31 | 23.09 ± 0.33 | 24.91 ± 0.35 | 25.87 ± 0.36 | 26.91 ± 0.40 |
| 8 | 19.11 ± 0.18 | 22.84 ± 0.27 | 24.34 ± 0.36 | 25.69 ± 0.31 | 26.88 ± 0.37 |
| 9 | 19.50 ± 0.23 | 23.15 ± 0.30 | 24.62 ± 0.37 | 25.85 ± 0.37 | 26.63 ± 0.38 |
| 10 | 19.69 ± 0.21 | 23.52 ± 0.23 | 24.89 ± 0.41 | 26.11 ± 0.45 | 27.71 ± 0.47 |
| 11 | 19.49 ± 0.18 | 22.73 ± 0.23 | 24.22 ± 0.28 | 25.78 ± 0.31 | 27.42 ± 0.36 |
| 12 | 19.36 ± 0.27 | 22.52 ± 0.31 | 24.31 ± 0.33 | 25.66 ± 0.38 | 27.69 ± 0.45 |
| 13 | 19.47 ± 0.32 | 22.07 ± 0.27 | 24.07 ± 0.31 | 25.15 ± 0.36 | 26.74 ± 0.41 |
| 14 | 19.81 ± 0.26 | 21.82 ± 0.33 | 23.74 ± 0.29 | 24.93 ± 0.27 | 26.93 ± 0.39 |
| 15 | 19.66 ± 0.19 | 21.94 ± 0.28 | 24.15 ± 0.35 | 25.07 ± 0.38 | 27.68 ± 0.44 |
| 16 | 19.32 ± 0.25 | 22.03 ± 0.24 | 24.38 ± 0.31 | 25.59 ± 0.29 | 27.81 ± 0.36 |
| 17 | 19.41 ± 0.17 | 22.29 ± 0.35 | 24.72 ± 0.29 | 26.01 ± 0.36 | 27.64 ± 0.42 |
| 18 | 19.65 ± 0.21 | 22.71 ± 0.28 | 25.65 ± 0.33 | 26.89 ± 0.45 | 28.38 ± 0.51 |

Compared with the blank group: ΔP < 0.05.

TABLE 2
The effect of the pharmaceutical composition of the present invention
on liver index and blood lipids in mice having a high-fat diet.

| | Total | | High density | | |
|---|---|---|---|---|---|
| | cholesterol | Triglycerides | lipoproteins | Liver wet | |
| Groups | (mmol/L) | (mmol/L) | (mmol/L) | weight (g) | Liver index |
| Blank | 3.47 ± 0.08 | 0.82 ± 0.04 | 2.26 ± 0.11 | 1.131 ± 0.073 | 4.16 |
| group | | | | | |
| Model | 5.39 ± 0.15ΔΔ | 1.44 ± 0.06Δ | 0.97 ± 0.08ΔΔ | 1.824 ± 0.134Δ | 6.26 ΔΔ |
| group | | | | | |
| 1 | 5.22 ± 0.07 | 1.27 ± 0.08 | 1.73 ± 0.09* | 1.619 ± 0.083 | 5.77 |
| 2 | 5.14 ± 0.13 | 1.13 ± 0.06 | 1.77 ± 0.14* | 1.434 ± 0.095* | 5.15 * |
| 3 | 5.08 ± 0.15 | 1.29 ± 0.04 | 1.74 ± 0.17* | 1.481 ± 0.117* | 5.26 * |
| 4 | 5.03 ± 0.08 | 1.11 ± 0.06 | 1.70 ± 0.15* | 1.495 ± 0.104 | 5.40 |
| 5 | 4.95 ± 0.10 | 1.35 ± 0.07 | 1.76 ± 0.13* | 1.467 ± 0.125* | 5.19 * |
| 6 | 5.01 ± 0.09 | 1.22 ± 0.06 | 1.79 ± 0.12* | 1.466 ± 0.083* | 5.30 * |
| 7 | 4.68 ± 0.12* | 1.26 ± 0.08 | 1.68 ± 0.16* | 1.483 ± 0.091* | 5.51 |
| 8 | 4.83 ± 0.10 | 1.12 ± 0.07 | 1.75 ± 0.10* | 1.473 ± 0.091* | 5.48 |
| 9 | 4.74 ± 0.08* | 1.25 ± 0.09 | 1.82 ± 0.13* | 1.480 ± 0.105* | 5.56 |
| 10 | 4.92 ± 0.13 | 1.31 ± 0.07 | 1.79 ± 0.10* | 1.492 ± 0.105 | 5.38 |
| 11 | 5.16 ± 0.17 | 1.25 ± 0.06 | 1.77 ± 0.13* | 1.503 ± 0.134 | 5.48 |
| 12 | 4.93 ± 0.09 | 1.16 ± 0.06 | 1.95 ± 0.07* | 1.489 ± 0.121* | 5.38 * |
| 13 | 4.14 ± 0.08** | 0.94 ± 0.05* | 2.14 ± 0.08** | 1.226 ± 0.135** | 4.58 ** |
| 14 | 4.86 ± 0.05* | 1.15 ± 0.07 | 1.88 ± 0.06* | 1.402 ± 0.114* | 5.21 * |
| 15 | 5.07 ± 0.11 | 1.20 ± 0.04 | 1.84 ± 0.13* | 1.469 ± 0.128* | 5.31 * |
| 16 | 5.03 ± 0.12 | 1.13 ± 0.06 | 1.79 ± 0.09* | 1.471 ± 0.104* | 5.29 * |
| 17 | 4.95 ± 0.09 | 1.22 ± 0.07 | 1.86 ± 0.14* | 1.483 ± 0.121* | 5.37 * |
| 18 | 5.20 ± 0.07 | 1.31 ± 0.07 | 1.64 ± 0.07* | 1.577 ± 0.149 | 5.56 |

Compared with the blank group: ΔP < 0.05; ΔΔP < 0.01; compared with the model group: *P < 0.05; **P < 0.01.

The experimental results in Tables 1-2 showed that compared to the blank group mice fed with standard diet, the model group mice on a high-fat diet had significantly increased body weight, liver wet weight, liver index, as well as serum total cholesterol and triglyceride levels, while the high-density lipoprotein level was significantly decreased, indicating the success of the hyperlipidemia model.

Chlorogenic acid and its combinations with vitamins and/or trace elements had shown certain inhibitory effects on the gain in body weight and liver weight of mice caused by a high-fat diet, as well as effects on reducing serum total cholesterol and triglyceride levels, together with effects on significantly increasing the level of high-density lipoprotein. This indicated that chlorogenic acid and its combinations with vitamins and/or trace elements had certain effects on reducing blood lipids. Moreover, the corresponding effect of the combinations of chlorogenic acid with vitamins and/or trace elements was generally better than that of chlorogenic acid alone, indicating that the combinations of chlorogenic acid with vitamins and/or trace elements had a significantly better lipid-lowering effect. However, the experimental results also showed that the more types of active ingredients in the pharmaceutical combination didn't always mean the stronger lipid-lowering effects. The reasons might include the influence of active substance water-solubility and different interactions among active substances, as well as the decrease in the concentration of a single active substance as increase in the active substance types, etc. After extensive screening experiments, it had been shown that under the same drug concentration, the combination of chlorogenic acid, vitamin C, and manganese gluconate had a significantly better lipid-lowering effect compared to other drug combinations (note: the lipid-lowering effect of chlorogenic acid and its combinations with vitamins and/or trace elements was only an example demonstration, and was not the complete experimental process of screening chlorogenic acid and its combinations with vitamins and/or trace elements in the present invention).

### Experimental Example 2: The Effect of the Ratio of Chlorogenic Acid, Vitamin C and Manganese Gluconate on the Lipid-Lowering Effect of the Pharmaceutical Compositions

### 2.1 Experimental Drugs

| Groups | Experimental drugs |
|---|---|
| 1 | chlorogenic acid:vitamin C:manganese gluconate = 100:9:0.09 |
| 2 | chlorogenic acid:vitamin C:manganese gluconate = 100:8:0.08 |
| 3 | chlorogenic acid:vitamin C:manganese gluconate = 100:7:0.07 |
| 4 | chlorogenic acid:vitamin C:manganese gluconate = 100:6:0.06 |
| 5 | chlorogenic acid:vitamin C:manganese gluconate = 100:5:0.05 |
| 6 | chlorogenic acid:vitamin C:manganese gluconate = 100:4:0.04 |
| 7 | chlorogenic acid:vitamin C:manganese gluconate = 100:3:0.03 |
| 8 | chlorogenic acid:vitamin C:manganese gluconate = 100:2:0.02 |
| 9 | chlorogenic acid:vitamin C:manganese gluconate = 100:1:0.01 |
| 10 | chlorogenic acid:vitamin C:manganese gluconate = |
| | 100:0.5:0.005 |

### 2.2 Experimental Methods

According to the method of Example 1, the effects of the combinations of chlorogenic acid, vitamin C and manganese gluconate in different ratios on the body weight, liver wet weight, liver index, and the levels of serum total cholesterols, triglycerides, and high-density lipoproteins of mice were determined. The specific experimental results are shown in Tables 3-4.

TABLE 3
The effect of the combination of chlorogenic acid, vitamin C and manganese
gluconate on the body weight of mice having a high-fat diet.

| | Body weight (g) | | | | |
|---|---|---|---|---|---|
| | Initial body | | | | |
| Groups | weight | Day 6 | Day 13 | Day 20 | Day 27 |
| Blank | 19.26 ± 0.12 | 20.72 ± 0.16 | 22.16 ± 0.23 | 23.63 ± 0.28 | 25.55 ± 0.34 |
| group | | | | | |
| Model | 19.37 ± 0.20 | 22.04 ± 0.28Δ | 24.82 ± 0.38Δ | 26.85 ± 0.33Δ | 29.35 ± 0.42ΔΔ |
| group | | | | | |
| 1 | 19.53 ± 0.18 | 21.59 ± 0.23 | 23.31 ± 0.22 | 25.01 ± 0.21 | 27.13 ± 0.46* |
| 2 | 19.49 ± 0.16 | 22.31 ± 0.35 | 24.28 ± 0.34 | 26.14 ± 0.28 | 27.67 ± 0.41 |
| 3 | 19.39 ± 0.24 | 21.94 ± 0.33 | 23.73 ± 0.34 | 25.54 ± 0.36 | 28.18 ± 0.41 |
| 4 | 19.62 ± 0.21 | 22.27 ± 0.26 | 24.15 ± 0.26 | 25.89 ± 0.42 | 27.67 ± 0.37 |
| 5 | 19.51 ± 0.18 | 21.81 ± 0.35 | 23.69 ± 0.37 | 25.93 ± 0.38 | 28.24 ± 0.41 |
| 6 | 19.60 ± 0.15 | 22.02 ± 0.37 | 23.94 ± 0.21 | 25.85 ± 0.20 | 27.66 ± 0.35 |
| 7 | 19.41 ± 0.23 | 21.35 ± 0.28 | 23.02 ± 0.29 | 24.55 ± 0.39* | 26.91 ± 0.40** |
| 8 | 19.58 ± 0.22 | 21.90 ± 0.31 | 23.61 ± 0.35 | 25.42 ± 0.37 | 27.28 ± 0.37 |
| 9 | 19.48 ± 0.19 | 21.87 ± 0.26 | 23.85 ± 0.28 | 25.69 ± 0.32 | 27.13 ± 0.38* |
| 10 | 19.39 ± 0.24 | 21.42 ± 0.27 | 23.26 ± 0.39 | 25.03 ± 0.39 | 27.71 ± 0.47 |

Compared with the blank group: ΔP < 0.05; ΔΔP < 0.01; compared with the model group: *P < 0.05.

TABLE 4
The effect of the combination of chlorogenic acid, vitamin C and manganese gluconate
on the liver index and blood lipids of mice having a high-fat diet.

| | Total | | High density | | |
|---|---|---|---|---|---|
| | cholesterol | Triglycerides | lipoprotein | Liver wet | |
| Groups | (mmol/L) | (mmol/L) | (mmol/L) | weight (g) | Liver index |
| Blank | 3.13 ± 0.06 | 0.80 ± 0.06 | 2.33 ± 0.15 | 1.120 ± 0.073 | 4.38 |
| group | | | | | |
| Model | 5.41 ± 0.27ΔΔ | 1.46 ± 0.08ΔΔ | 0.91 ± 0.12ΔΔ | 1.917 ± 0.155ΔΔ | 6.53 ΔΔ |
| group | | | | | |
| 1 | 5.06 ± 0.19 | 1.20 ± 0.13 | 1.96 ± 0.17** | 1.737 ± 0.093 | 6.40 |
| 2 | 4.96 ± 0.15* | 1.15 ± 0.07 | 1.83 ± 0.11** | 1.631 ± 0.117* | 5.89 |
| 3 | 5.01 ± 0.21 | 1.18 ± 0.09 | 1.95 ± 0.21** | 1.662 ± 0.154* | 5.90 |
| 4 | 4.83 ± 0.13* | 1.01 ± 0.14* | 2.02 ± 0.13** | 1.573 ± 0.161* | 5.68 |
| 5 | 4.72 ± 0.18* | 1.08 ± 0.06* | 1.94 ± 0.18** | 1.491 ± 0.108** | 5.28 * |
| 6 | 4.53 ± 0.20** | 0.96 ± 0.10** | 2.07 ± 0.23** | 1.407 ± 0.118** | 5.09 * |
| 7 | 4.25 ± 0.16** | 0.89 ± 0.07** | 2.26 ± 0.15** | 1.256 ± 0.142** | 4.67 ** |
| 8 | 4.67 ± 0.23* | 0.94 ± 0.12** | 2.09 ± 0.18** | 1.419 ± 0.101** | 5.20 * |
| 9 | 4.82 ± 0.11* | 1.15 ± 0.13 | 1.99 ± 0.16** | 1.527 ± 0.134* | 5.63 |
| 10 | 5.14 ± 0.26 | 1.26 ± 0.13 | 1.72 ± 0.07** | 1.791 ± 0.125 | 6.46 |

Compared with the blank group: ΔP < 0.05; ΔΔP < 0.01; compared with the model group: *P < 0.05; **P < 0.01.

The experimental results in Tables 3-4 showed that the ratio of chlorogenic acid, vitamin C and manganese gluconate had a significant impact on the lipid-lowering effect. With the decrease of the ratio of vitamin C and manganese gluconate, the lipid-lowering effect of the pharmaceutically combination gradually increased. When chlorogenic acid:vitamin C:manganese gluconate=100:3:0.03, the corresponding combination showed the optimal lipid-lowering effect, and then with the further decrease of the ratio of vitamin C and manganese gluconate, the lipid-lowering effects of corresponding combinations gradually decreased (note: the above ratio of chlorogenic acid, vitamin C, and manganese gluconate was only an example demonstration, and was not the complete experimental process of screening the ratio of chlorogenic acid, vitamin C, and manganese gluconate in the present invention)

The above had described the preferable examples of the present invention, which were not to be construed as a limitation of the scope of the present invention. Those skilled in the art may make various improvements and modifications on the embodiments disclosed herein, without departing from the scope and basic spirit of the present invention.

1. A pharmaceutical composition for treating hyperlipidemia, characterized in that it comprises chlorogenic acid, vitamins, pharmaceutically acceptable excipients, and optional trace elements.

2. The pharmaceutical composition for treating hyperlipidemia according to claim 1, characterized in that the pharmaceutical composition for treating hyperlipidemia comprises chlorogenic acid, vitamins, and optional trace elements as the only active ingredients.

3. The pharmaceutical composition for treating hyperlipidemia according to claim 1, characterized in that the vitamin is selected from: vitamin C, vitamin E, vitamin B6, vitamin B12 or a combination thereof; preferably, the vitamin is selected from vitamin C.

4. The pharmaceutical composition for treating hyperlipidemia according to claim 1, characterized in that the trace element is selected from: zinc, manganese, selenium, chromium, copper or a combination thereof; preferably, the trace element is selected from zinc gluconate, manganese gluconate, chromium chloride, copper gluconate or a combination thereof; more preferably, the trace element is selected from manganese gluconate.

5. The pharmaceutical composition for treating hyperlipidemia according to claim 1, characterized in that the weight ratio of chlorogenic acid, vitamins, and optional trace elements is 100:(1-5):(0.01-0.05); preferably, the weight ratio of chlorogenic acid, vitamins, and optional trace elements is 100:(2-4):(0.02-0.04); more preferably, the weight ratio of chlorogenic acid, vitamins, and optional trace elements is 100:3:0.03.

6. The pharmaceutical composition for treating hyperlipidemia according to claim 1, characterized in that the pharmaceutical composition for treating hyperlipidemia is administered by a gastroenteric or parenteral route, and preferably, the pharmaceutical formulation for gastrointestinal administration includes tablets, capsules, granules, and oral liquids, while the pharmaceutical formulation for parenteral administration includes injection, infusion solution, and freeze-dried powder injections.

7. The preparation method of the pharmaceutical composition for treating hyperlipidemia according to claim 1, characterized in that it comprises the following steps:
(1) Each raw material is weighed according to the pre-determined amount;
(2) A pharmaceutical formulation for administration by the gastrointestinal or parenteral pathways is prepared according to conventional methods in this field.

8. The combination of chlorogenic acid, vitamins, and optional trace elements for use in the manufacture of a pharmaceutical composition for treating hyperlipidemia.

9. The use according to claim 8, characterized in that the vitamin is selected from: vitamin C, vitamin E, vitamin B6, vitamin B12 or a combination thereof, and the trace element is selected from: zinc, manganese, selenium, chromium, copper or a combination thereof.

10. The use according to claim 9, characterized in that the weight ratio of chlorogenic acid, vitamins, and optional trace elements is 100:(1-5):(0.01-0.05).
